(19) United States
(12) Patent Application Publication
(10) Pub. No.: US 2014/0121260 A1
(43) Pub. Date: May 1, 2014

(54) NOVEL CONJUGATES OF POLYUNSATURATED FATTY ACIDS WITH AMINES AND THERAPEUTIC USES THEREOF

(71) Applicant: MedWell Laboratories Ltd., Nazareth (IL)

(72) Inventors: Taher NASSAR, Tur'an (IL); Raiyn Nureldin, Kfar-Kabul (IL); Rayan Anwar, Kfar-Kabul (IL)

(73) Assignee: MedWell Laboratories Ltd., Nazareth (IL)

(21) Appl. No.: 14/147,821

(22) Filed: Jan. 6, 2014

Related U.S. Application Data
(63) Continuation of application No. 12/519,553, filed on Feb. 25, 2010.
(60) Provisional application No. 60/875,832, filed on Dec. 20, 2006.

(51) Int. Cl. A61K 47/48

(52) U.S. Cl. CPC A61K 47/48038; A61K 47/48061
USPC 514/422; 514/423; 548/533; 548/527

(57) ABSTRACT

The present application discloses conjugates comprising an aminic moiety covalently linked to a hydrophobic moiety, through an amidic bond, which are selective inhibitors of COX-2 enzyme and can be beneficially used in the treatment of various inflammatory disease or disorder and cancer.

### CROSS-REFERENCE TO RELATED APPLICATIONS

The present application is a Continuation Application of U.S. application Ser. No. 12/519,553 filed on Feb. 25, 2010 which is a National Phase application of PCT International Application no. PCT/IL2007/001592 filed on Dec. 20, 2007 claiming the benefit of U.S. Provisional Patent Application No. 60/875,832, filed on Dec. 20, 2006, all of which are hereby incorporated by reference.

### TECHNICAL FIELD

The present invention relates to pharmaceutical substances and compositions in general and more particularly, to pharmaceutical substances and compositions useful inter alia in inhibiting cyclooxygenase enzymes, treating inflammations, rheumatoid arthritis, inflammatory bowel diseases such as colitis and Crohn's disease, asthma, autoimmune diseases, chronic inflammations, chronic prostatitis, glomerulonephritis, hypersensitivities, pelvic inflammatory disease, reperfusion injuries, transplant rejections, vasculitis, diabetes, cardiovascular disorder, pathogenically induced inflammations and cancer.

### BACKGROUND ART

Inflammation is the complex biological response of vascular tissues to harmful stimuli, such as pathogens, damaged cells, or irritants. It is a protective attempt by the organism to remove the injurious stimuli as well as initiate a healing process of the tissue. Inflammation is not a synonym for infection. Even in cases where inflammation is caused by infection it is incorrect to use the terms as synonyms: infection is caused by an exogenous pathogen, while inflammation is the response of the organism to the pathogen.

In the absence of inflammation, wounds and infections would never heal and progressive destruction of the tissue would compromise the survival of the organism. However, inflammation which runs unchecked can also lead to a host of diseases, such as high fever, atherosclerosis, and rheumatoid arthritis. It is for this reason that inflammation is normally tightly regulated by the body.

Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

Inflammatory disorders are abnormalities associated with inflammation and comprise a large, unrelated group of disorders which underlie a variety of human diseases. The immune system is often involved with inflammatory disorders, demonstrated in both allergic reactions and some myopathies, with many immune system disorders resulting in abnormal inflammation. Non-immune diseases with aetiological origins in inflammatory processes are thought to include cancer, atherosclerosis, and ischaemic heart disease.

A large variety of proteins are involved in inflammation, and any one of them is open to a genetic mutation which impairs or otherwise dysregulates the normal function and expression of that protein.

Examples of disorders associated with inflammation include:

rheumatoid arthritis, inflammatory bowel diseases such ascolitis and Crohn's disease, asthma, autoimmune diseases, chronic inflammations, chronic prostatitis, glomerulonephritis, hypersensitivities, pelvic inflammatory disease, reperfusion injuries, transplant rejections, vasculitis, diabetes, cardiovascular disorder and pathogenically induced inflammations.

Nonsteroidal anti-inflammatory drugs (NSAIDs) such as aspirin exhibit their anti-inflammatory effect by inhibiting cyclooxygenase (COX) which catalyzes the first step in arachidonic acid metabolism. It was realized in the late 1980s that the isozyme COX-1 is constitutive and responsible for the physiological production of prostaglandins, while COX-2 is inducible and responsible for the elevated production of prostaglandins during inflammation. COX-3, another isozyme of COX, was also reported recently. The chronic use of NSAIDs to treat pain and inflammation is often accompanied by side effects such as gastric ulceration, bleeding, and renal function suppression. This was the fundamental rationale for the development of selective inhibitors of the COX-2 enzyme as a new class of anti-inflammatory and analgesic agent with improved gastrointestinal tolerability. However, the potential of this new class of drugs has not been realized, even though the rationale underpinning their use is likely to be correct.

COX-2 was discovered in the early 1990s, and a large amount of effort has been expended since then by the pharmaceutical industry in the search for COX-2 inhibitors. In 2000, COX-2 inhibitors were involved in clinical trials for the treatment of colon and rectal cancers because COX-2 over-expression had been found in malignant tumor samples.

However, at this point, the risk of heart attack and stroke emerged, and this is possibly because COX-2 is physiologically expressed in the glomeruli and cortex, and COX-2 may also have an anti-inflammatory role. Recently, a number of events regarding COX-2 inhibitors have attracted the attention of the media. Rofecoxib (Vioxx,) was withdrawn by Merck & Co in September 2004 following the finding from the Vioxx Gastrointestinal Outcomes Research (VIGOR) trialfor colon and rectal cancers that there were severe risks for the heart. Similar claims were made when a COX-2 inhibitor was used in the adenoma prevention with celecoxib (APC) trial, which was eventually suspended in December 2004 because analysis by an independent Data Safety and Monitoring Board showed that there was a risk of fatal and nonfatal major cardiovascular disease (CVD).

On Feb. 20, 2005, a US Food and Drug Agency (FDA) advisory panel unanimously concluded that all classes of COX-2 inhibitors increase a person's risk of heart attack and stroke, and recommended that, despite the risks, drugs including Vioxx should remain on the market but be accompanied by strong warnings. Following the FDA's decision, the European Union and countries in which COX-2 inhibitors were used imposed similar restrictions. Because the drugs are now “tainted”, physicians are reluctant to prescribe them, and patients do not want to risk the health of their heart to ease pain, the sales of Celebrex, valdecoxib and rofecoxib have dropped dramatically. As COX-2 inhibitors have become controversial, many pharmaceutical companies have to renew their research efforts in the search for new effective anti-inflammatory drugs to treat arthritis and other inflammatory diseases.

This is a fundamental rationale for the development of new, safe, non-toxic selective inhibitors of the COX-2 enzyme as a new class of anti-inflammatory and analgesic agent with improved gastrointestinal tolerability.

### SUMMARY OF THE INVENTION

The present invention is based on the findings that conjugates of certain organic molecules, such as proline and structural analogs and derivatives thereof such as taurine and other small organic molecules, and a hydrophobic moiety, such as saturated and/or unsaturated fatty acid and/or a high alkyl (e.g., having at least 6 carbon atoms in its chain), optionally substituted, are efficient selective inhibitors of COX-2 and hence can be beneficially utilized in the treatment of pain (as analgesics) and inflammations, rheumatoid arthritis, inflammatory bowel diseases such as colitis and Crohn's disease, asthma, autoimmune diseases, chronic inflammations, chronic prostatitis, glomerulonephritis, hypersensitivities, pelvic inflammatory disease, reperfusion injuries, transplant rejections, vasculitis, diabetes, cardiovascular disorder and pathogenically induced inflammations.

Moreover, recent discoveries showing the potency of NSAIDs and especially of COX-2 inhibitors in treatment and prevention of cancers of the colon, esophagus, lung, bladder, breast and prostate and hence can be used for treatment thereof; COX-2 inhibitors in cancer treatment and prevention, a recent development, Anticancer Drugs, 2002 February;13(2):127-37; Cyclooxygenase-2: a potential target in breast cancer, Seminars in Oncology, 2004 February;31(1 Suppl 3):64.; Lung cancer and cyclooxygenase-2, The Annals of Thoracic Surgery, 2003 October;76(4):1327-35. and HER2 and COX2 expression in human prostate cancer, European Journal of Cancer, 2004 January;40(1):50-5.

### BRIEF DESCRIPTION OF THE DRAWINGS

The present invention will be understood and appreciated more fully from the following detailed description taken in conjunction with the appended drawings in which:

FIGS. 1 A-O are diagrams of the aminic moieties of the conjugates of the present invention;

FIGS. 2 A-F are diagrams of the hydrophobic moieties of the conjugates of the present invention;

FIGS. 3 A-D are diagrams of several exemplary conjugates;

FIGS. 4 A-F are diagrams of exemplary conjugates that were synthesized according to a method disclosed herein;

FIGS. 5 A-F are diagrams of exemplary conjugates that were subjected to in-vitro and in-vivo studies;

FIGS. 6 A and B are bar charts of the empirical data of in-vivo study of the effect of the RNX003 compound on the PGE2 production and the levels of TNFα in CIA mice;

FIG. 7 is a bar charts of the empirical data of in-vivo study of the effect of the effect of the RNX006 compound on experimentally induced ulcerative colitis in rats compared to the effect of 5-ASA;

FIG. 8 is a chart of cell viability plotted versus the increasing concentrations of the RNX003 compound of MTT test.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention.

### DISCLOSURE OF THE INVENTION

Illustrative embodiments of the invention are described below. It will be appreciated that in the development of any such actual embodiment, numerous implementation-specific decisions must be made to achieve the developers' specific goals, such as compliance with system-related and business-related constraints, which vary from one implementation to another. Moreover, it will be appreciated that such a development effort might be complex and time-consuming, but would nevertheless be a routine undertaking for those of ordinary skill in the art having the benefit of this disclosure.

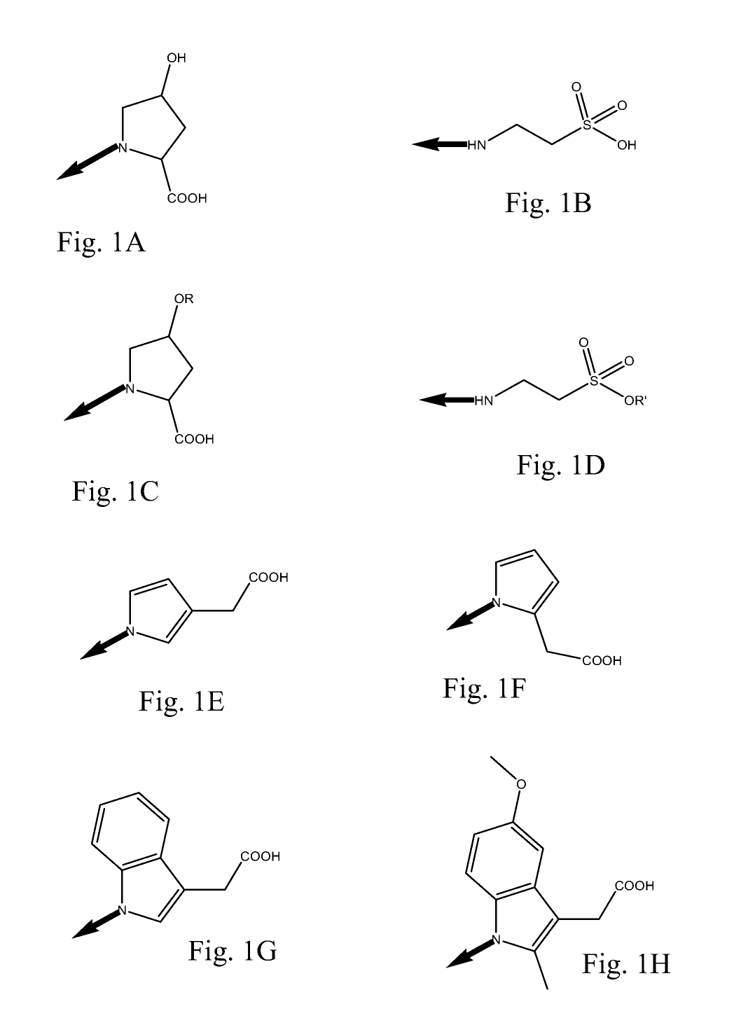

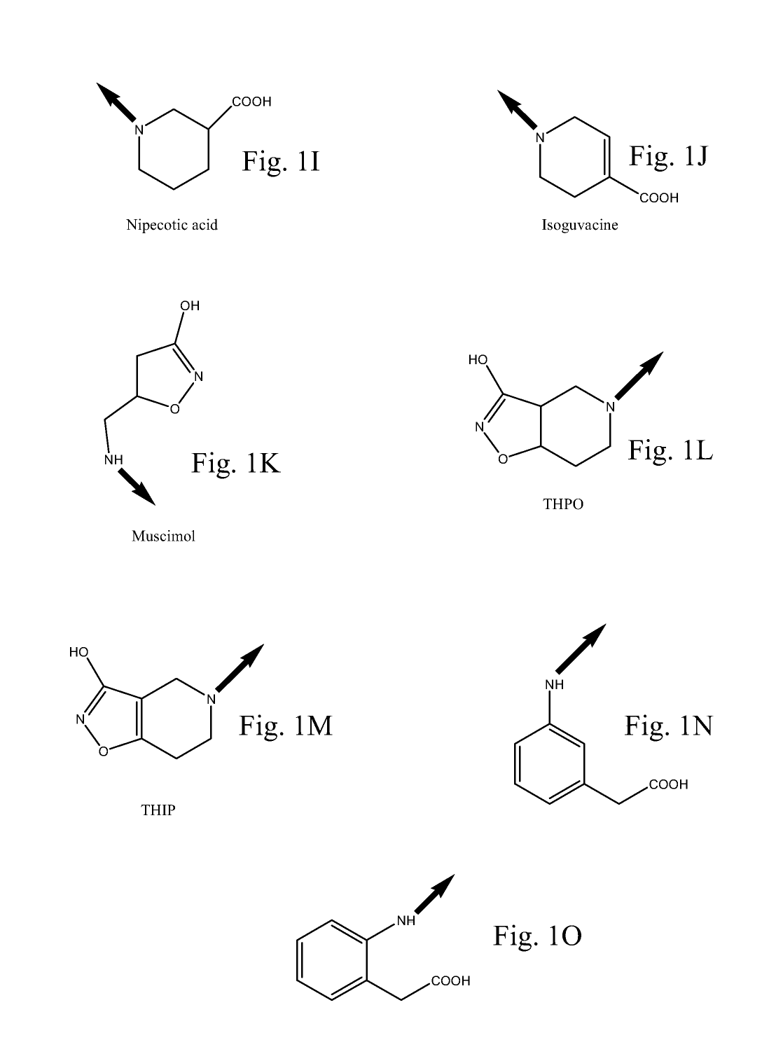

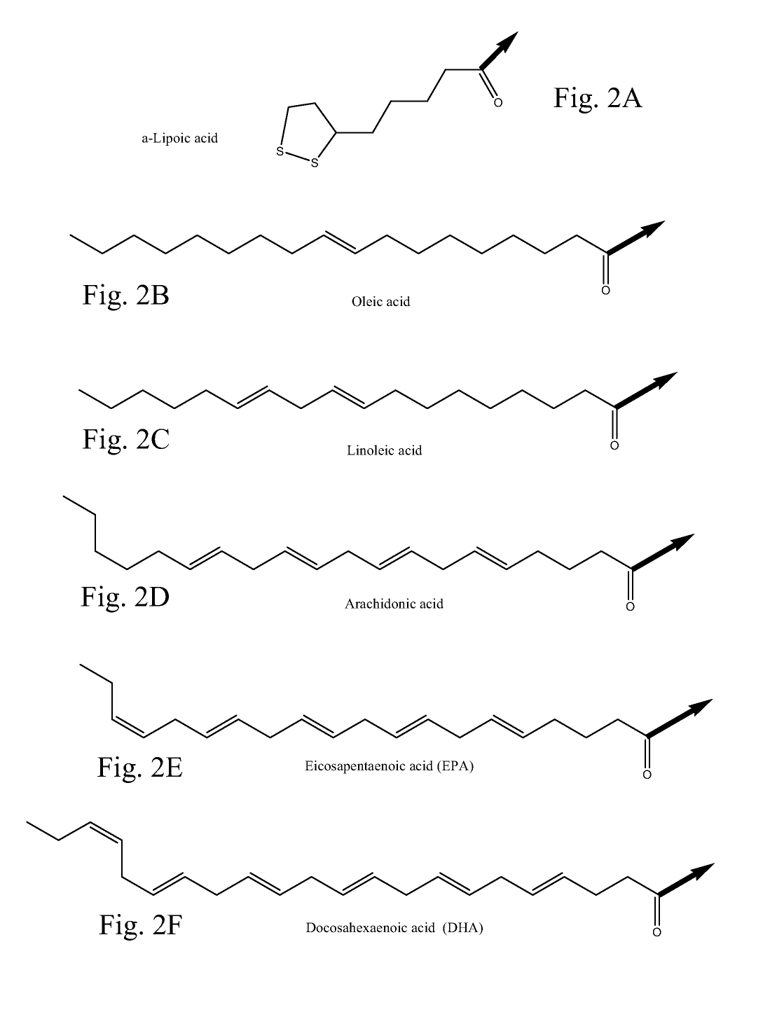

The conjugates according to the present invention can comprise any combination of the two moieties, M1 and M2, selected from the two respective groups shown in FIGS. 1 A-O and FIGS. 2 A-F, to which reference is now made. The aminic moieties of the M1 group, shown in FIGS. 1 A-O, are characterized by a terminal amine which is to form an amidic bond (indicated by arrow), of either a secondary or tertiary amide, with a terminal carbonyl (indicated by a receptive arrow) of the hydrophobic moieties of the M2 group, shown in FIGS. 2 A-F, thereby forming the conjugates of the present invention. The aminic moieties of the M1 group, shown in FIGS. 1 C and D, respectively incorporate R and R′ residues; wherein R is hydrogen, an alkyl composed of up to 30 carbon atoms or an acyl composed of up to 30 carbon atoms, and R′ is hydrogen or an alkyl composed of up to 30 carbon atoms. The hydrophobic moieties of the M2 group are a-Lipoic acid, Oleic acid, Linoleic acid, Arachidonic acid, Eicosapentaenoic acid (EPA) and Docosahexaenoic acid (DHA).

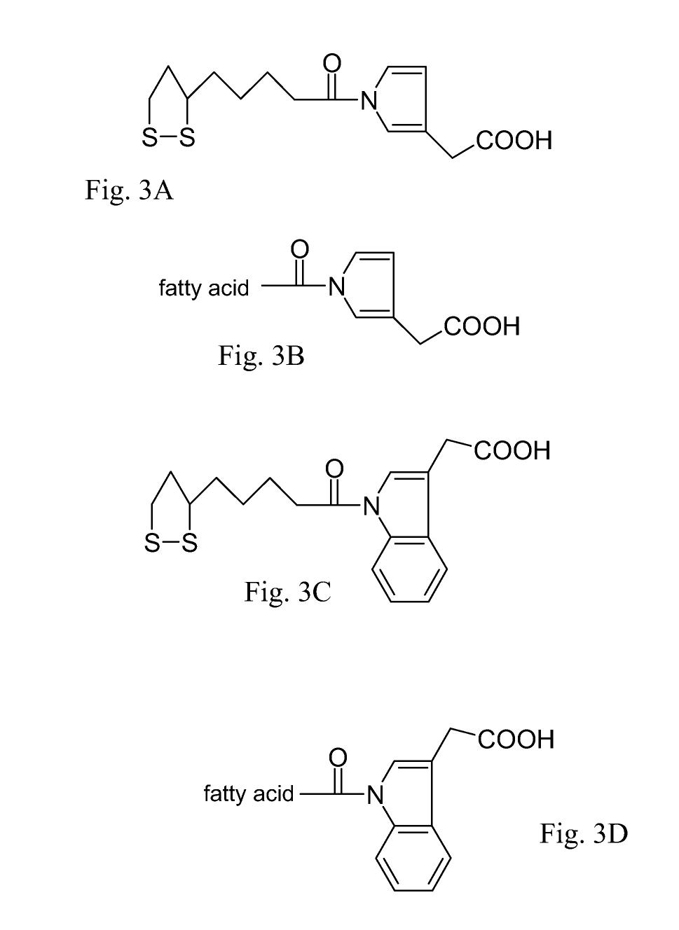

Reference is now made to FIGS. 3 A-D, in which chemical structures of exemplary conjugates in accordance with the present invention are sown.

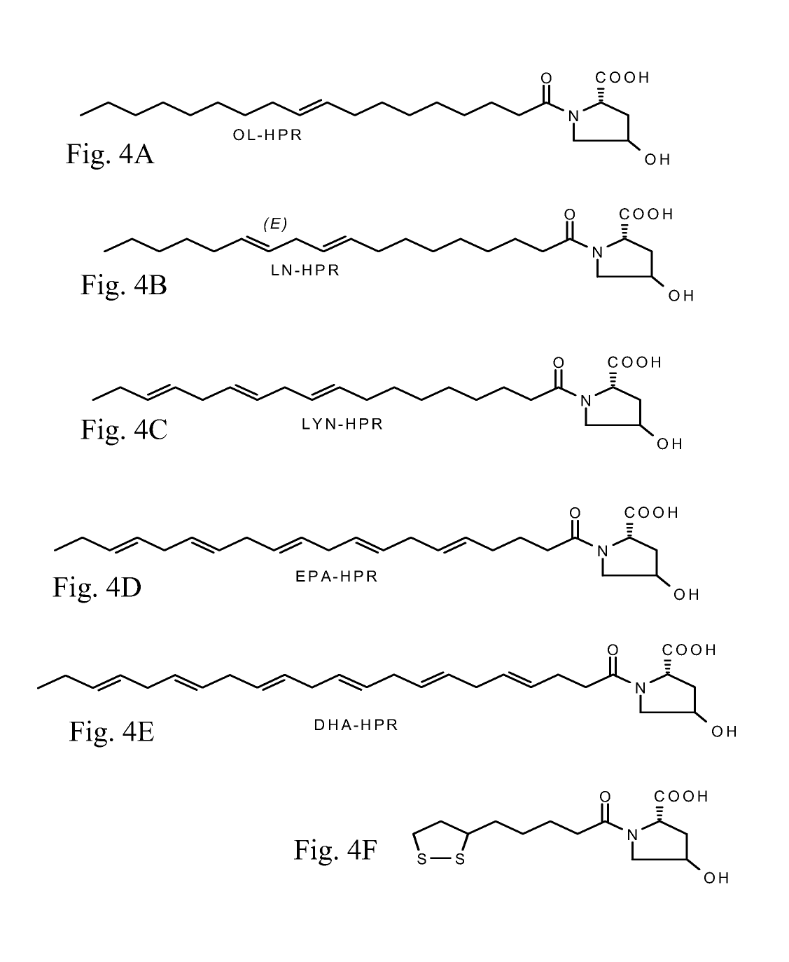

The exemplary conjugates in FIGS. 4 A-F, to which reference is now made, were synthesized according to the procedure elaborated infra A solution of fatty acid (FA) (1.5 mol) and N-hydroxysuccinimide (1.6 mmol) in dry dichloromethane is cooled in an ice-water bath and dicylohexylcarbomiimide (1.6 mol) is added with stirring. The mixture is kept in the refrigerator (between 0 and +5° C.) overnight. The separated N,N′-dicyclohexylurea is removed by filtration and the solvent evaporated in vacuum. The crude product is twice recrystalized from isopropanol.

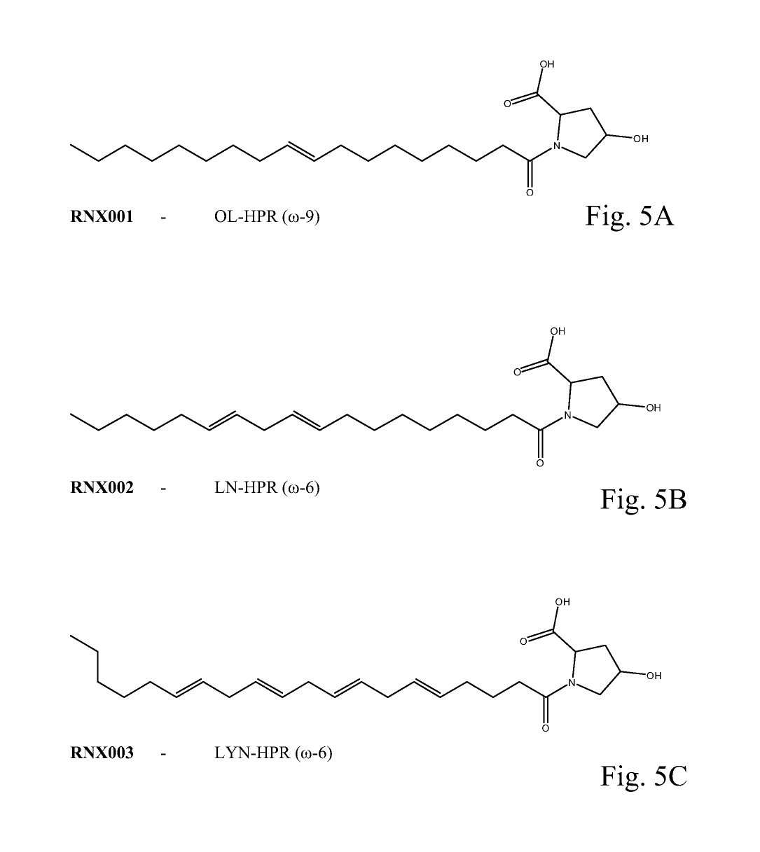

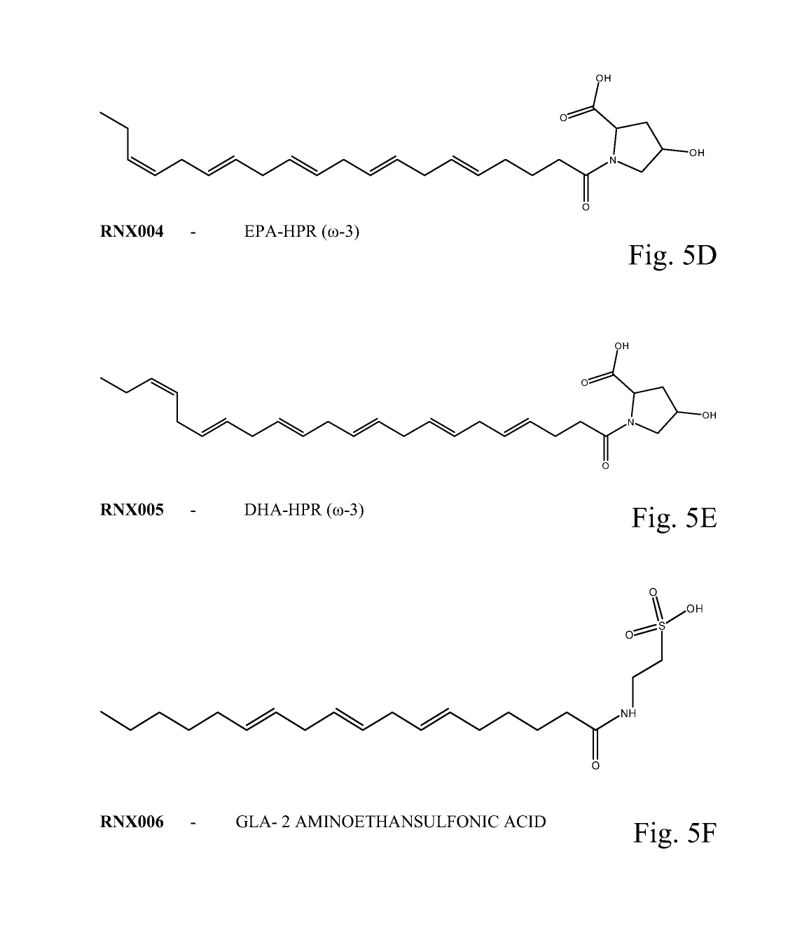

The exemplary conjugates shown in FIGS. 5 A-E, to which reference is now made, were tested for inhibition of COX-1 and COX-2 to in-vitro activity The ability of the test compounds listed in the Table 1 to inhibit ovine COX-1 and COX-2 (IC50 values, mM) was determined using an enzyme immuno assay (EIA) kit catalog number 560101, Cayman Chemical, Ann Arbor, Mich., USA) according to the manufacturers instructions.

Cyclooxygenase catalyzes the first step in the biosynthesis of arachidonic acid (AA) to PGH2. PGF2α, produced from PGH2 by reduction with stannous chloride, is measured by enzyme immunoassay (ACETM competitive EIA).

Stock solutions of test compounds were dissolved in a minimum volume of DMSO. Briefly, to a series of supplied reaction buffer solutions (960 μL, 0.1M Tris-HCl pH 8.0 containing 5 mM EDTA and 2 mM phenol) with either COX-1 or COX-2 (10 μL) enzyme in the presence of heme (10 μL) were added 10 μL of various concentrations of test drug solutions (0.01, 0.1, 1, 10, 50, and 100 μM in a final volume of 1 mL). These solutions were incubated for a period of 5 min at 37° C. after which 10 μL of AA (100 μM) solution were added and the COX reaction was stopped by the addition of 50 μL of 1M HCl after 2 min. PGF2α, produced from PGH2 by reduction with stannous chloride was measured by enzyme immunoassay.

This assay is based on the competition between PGs and a PG-acetylcholinesterase conjugate (PG tracer) for a limited amount of PG antiserum. The amount of PG tracer that is able to bind to the PG antiserum is inversely proportional to the concentration of PGs in the wells since the concentration of PG tracer is held constant while the concentration of PGs varies. The concentration of the test compound causing 50% inhibition (IC50, μM) was calculated from the concentration-inhibition response curve (duplicate determinations).

This assay is based on the competition between PGs and a PG-acetylcholinesterase conjugate (PG tracer) for a limited amount of PG antiserum. The amount of PG tracer that is able to bind to the PG antiserum is inversely proportional to the concentration of PGs in the wells since the concentration of PG tracer is held constant while the concentration of PGs varies. The concentration of the test compound causing 50% inhibition (IC50, μM) was calculated from the concentration-inhibition response curve (duplicate determinations).

TABLE 1
In vitro COX-1 and COX-2 activity inhibition by the exemplary
conjugates shown in FIGS. 5 A-E

IC50 (μM)

| Compound | COX-1 | COX-2 | Ration (COX ½) |
|---|---|---|---|
| RNX001 | 72.45 | 1.3 | 55.7 |
| OL-HPR | | | |
| RNX002 | 52.68 | 0.51 | 103.3 |
| LN-HPR | | | |
| RNX003 | 45.25 | 0.14 | 323.21 |
| LYN-HPR | | | |
| RNX004 | 39.45 | 0.085 | 464.11 |
| EPA-HPR | | | |
| RNX005 | 42.82 | 0.081 | 528.68 |
| DHA-HPR | | | |

The ability of the exemplary conjugates shown in FIGS. 5 A-E to selectively inhibit the in-vitro activity of COX-2 relatively to COX-1 was confirmed by the results of in-vitro studies shown supra.

The exemplary conjugates shown in FIGS. 5 A-C were thence subjected to in-vivo study of paw edema measurements in rats. Sprague dawley rats (150-200 g) were used. Edema was induced by a single sub-plantar injection of carrageenin (1 mg/paw) into the left hind paw of the rat under light ether anesthesia. The total volume injected was always 0.1 ml. The paw volume was measured immediately before the injection and at hourly intervals thereafter using a hydroplethysmometer (model 7150, Ugo Basile, Italy). The results were expressed either as the increase in paw volume (ml) calculated by subtracting the basal volume or by calculating the area under the time-course curve (AUC; ml h) for each group.

The anti-inflammatory activity of the compounds was tested versus Ibuprofen, as a reference, on carrageenan-induced edema at different time intervals as depicted in Table 2. After 1 h, all the tested hydroxyproline-FA amides compounds showed a reasonable decrease in the edema size ranging between 31% for compound OL-HPR and 43.4% for compound LN-HPR. This reduction considered very significant from that of control as shown in Table 2, the LYN-HPR derivative was the most potent anti-inflammatory compound after 4 hours, compared to ibuprofen. After 4 hours, the inflammation reached the highest size (3.1) and all the tested compounds were able to decrease the paw edema. Compound LYN-HPR was the most powerful one in minimizing the inflammation size (52% after 4 h).

TABLE 2
Inhibitory effect the exemplary conjugates shown in FIGS. 5 A-C on
carrageenin-induced edema of the hind paw in rats.

| | Swelling volume (ml) | | | |
|---|---|---|---|---|
| Compound | 1 hr | 2 hr | 3 hr | 4 hr |
| Control | 1.6 ± 0.08 | 2.9 ± 0.1 | 3.2 ± 0.12 | 3.1 ± 0.2 |
| Ibuprofen | 1.2 ± 0.2 | 1.9 ± 0.3 | 2.3 ± 0.16 | 2.4 ± 0.19 |
| RNX001 | 1.1 ± 0.09 | 1.3 ± 0.13 | 1.9 ± 0.1 | 1.8 ± 0.13 |
| OL-HPR | | | | |
| RNX002 | 0.9 ± 0.06 | 1.6 ± 0.09 | 2.3 ± 0.11 | 2.1 ± 0.13 |
| LN-HPR | | | | |
| RNX003 | 1.1 ± 0.11 | 1.4 ± 0.08 | 1.4 ± 0.09 | 1.5 ± 0.14 |
| LYN-HPR | | | | |

The correlation between the results of in vitro studies of COX-2 versus COX-1 selective activity inhibition by the exemplary conjugates shown in FIGS. 5 A-C and the in-vivo inhibitory effect thereof on carrageenin-induced edema of the hind paw in rats asserts that the analgesic/antipyretic/anti-inflammatory activity thereof is achieved through inhibition of cyclooxygenase-2 (COX-2) via inhibition of prostaglandin synthesis.

The RNX003 compound shown in FIG. 5C was subjected to further in-vivo study of PGE2 production and TNFα levels in collagen induced arthritis (CIA) in mice. Male black/57 mice, age 8-10 weeks, were used for the study of CIA. Bovine CII (type II collagen CII, Sigma, St. Louis, Mo., USA) was dissolved in 0.1 M acetic acid overnight at 4° C. This was emulsified in an equal volume of complete Freund's adjuvant (Sigma). The mice were immunized intradermally at the base of the tail with 100 μl of emulsion containing 100 μg of CII. On day 21, mice were boosted intraperitoneally with 100 μg CII dissolved in phosphate buffered saline (PBS). This model has been widely used to study disease mechanisms and potential therapies for RA. Indeed, CIA model has many morphological features similar to those of human RA including patterns of synovitis, pannus formation, and erosion of articular cartilage and bone. Moreover, CIA shares with RA many of the cytokines and biological factors in the synovium and cartilage.

RNX003 (purity>96.91%) was synthesized for drug treatment according to a procedure similar to the one elaborated supra. Ibuprofen was purchased from Sigma (Rehevot, Israel). Both were dissolved in 80% Cremophor EL: saline 80%:20% respectively. In order to assess the effect of RNX003 on the established CIA, treatment was commenced from the first day of the onset of the clinical symptoms of arthritis, which was considered to be the day when the first visible signs of erythema and/or oedema were observed in any of the limbs. Mice were randomly selected and assigned to one of the following groups: RNX003 (250 mg/(kg/day); n=4), Iboprofen (500 mg/(kg/day); n=4) or vehicle (n=4). The route of RNX003 and Ibuprofen delivery was oral administration. Treatment was given daily for a period of 21 days.

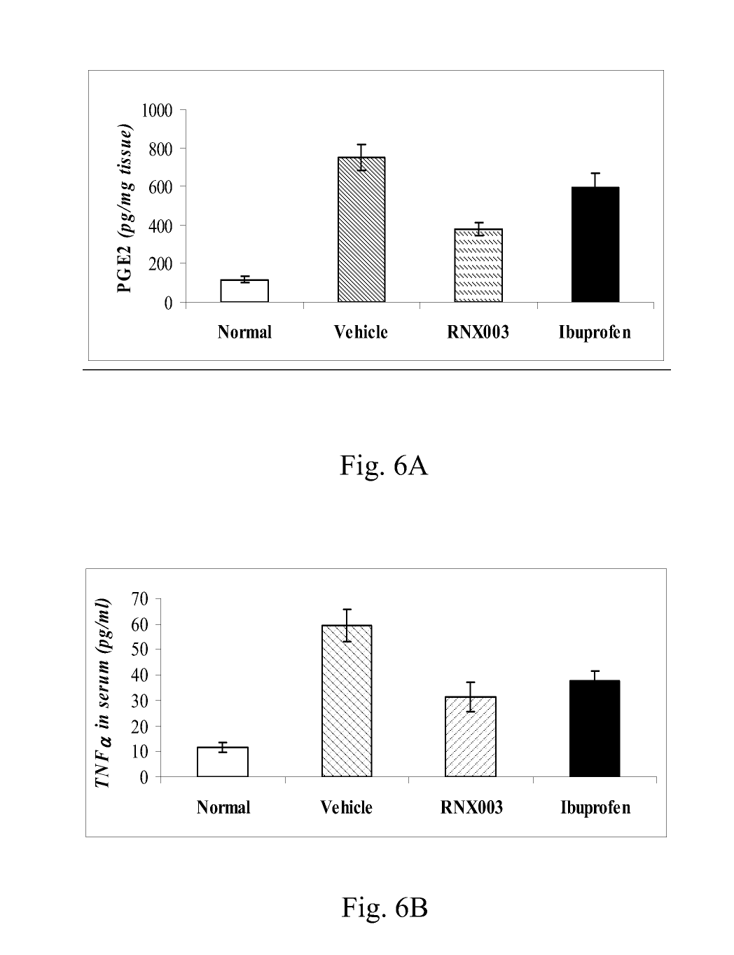

The assay for the production of PGE2 and TNFα was performed as elaborated infra. Joint tissues were prepared as previously described for measuring the production of PGE2 and cytokines Briefly, the left forepaw (including the paw, ankle, and knee) from each mouse was removed and homogenized in 100 mg tissue/1 ml of lysis medium (75% ethanol in 0.1 M sodium acetate, adjusted to pH 3 with HCl for PGE2, and RPMI 1640 containing 2 mM phenylmethylsulfonyl fluoride and 1 mg/ml of aprotinin, leupeptin, and pepstatin A for cytokines). The homogenates were then centrifuged 3500×g for 15 min at 4° C. Sera were obtained from the mice on day 22 of arthritis, as described above. Supernatants and sera were stored at −20° C. until use. PGE2 concentration was measured with a commercial radio immunoassay (RIA) kit (Amersham, UK) according to the manufacturer's instructions. Commercial enzyme-linked immunosorbent assay kit was used to measure the concentrations of TNFa (Diaclone, France) in serum according to the manufacturer's instructions. Results were expressed as pg/ml of serum or supernatant from joint homogenate as shown in FIGS. 6 A and B, to which reference is now made.

The exemplary RNX006 compound shown in FIG. 5 F, to which reference is now made, was tested for the effect on the inflammatory bowel disease IBD (ulcerative colitis) in rats as follows. Male Sprague dawley rats were used in this study. Briefly, ulcerative colitis was induced by tri-nitrobenzene sulfunic acid as described in the literature. Rats were divided into 4 groups, (−) sham (healthy, (+) control ulcerative colitis (UC, not treated), UC treated with RNX006 (25 mg/kg), and UC treated with 5-amino salicylic acid (5-ASA 25 mg/kg). RNX006 and 5-ASA were administered rectally during all the period of the experiment. At the end of the experiment animals were sacrificed and the colon was isolated to test the severity of the inflammation. The severity of the inflammation was tested by measurement of the myeloperoxidase activity (MPO activity) in the inflamed area of the colon.

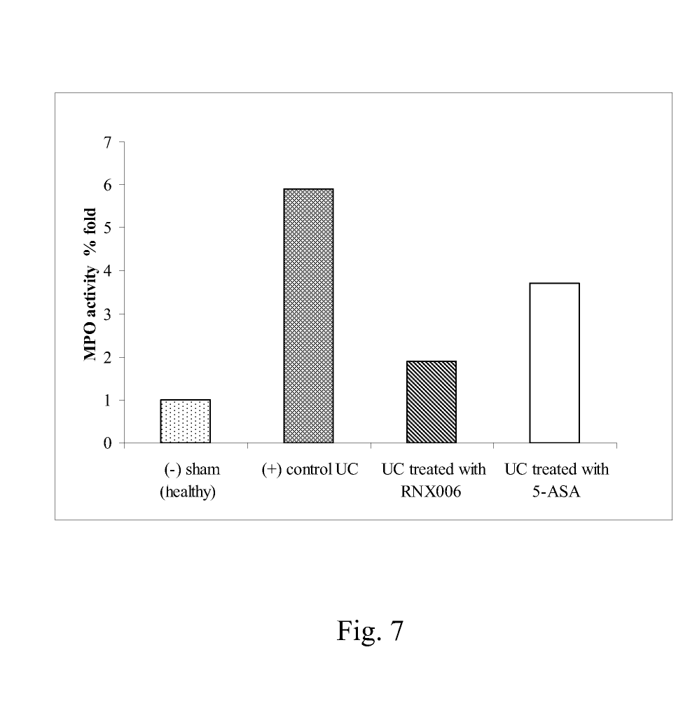

Reference is now made to FIG. 7 showing the results of the RNX006 compound effect on the inflammatory bowel disease IBD (ulcerative colitis) in rats. The IBD rats treated with the RNX006 compound exhibited significantly lower MPO activity relatively to the (+) control of non-treated IBD rats and even a lower MPO activity relatively IBD rats treated with 5-ASA. These results indicate that the RNX006 compound is a more potent therapeutic agent than 5-ASA in treatment of IBD since it is able to reduce the levels of MPO activity closely to those of (−) sham control.

The cytotoxic effects of the RNX003 compound, shown in FIG. 5C, were then evaluated using MTT test in a cell culture system using human keratinocytes cell line Hacat. Hacat cell line retains differentiated keratinocyte phenotype and can be grown indefinitely, thus permitting long-term studies to be performed. Cells were grown in Dulbecco's modified Eagle's medium (DMEM) with a high glucose content (4.5 g/L) supplemented with 10% vol/vol inactivated fetal calf serum, 1% nonessential amino acids, 1% glutamine, 100 U/mL penicillin, and 10 μg/mL streptomycin. Cells were maintained in humidified atmosphere of 95% O2-5% CO2 at 37° C. The pH of the media was monitored at 7.4. The medium of cells from both cell lines was changed twice a week. At 70-80% confluence, cells were trypsinized and plated in 96 well-microtiter plates. 24 h after cell seeding cells were exposed to various concentrations of RNX003

MTT assay was performed as follows. The tetrazolium dye, MTT, is widely used to assess the viability and/or the metabolic state of the cells. This colorimetric assay is based on the conversion of the yellow tetrazolium bromide (MTT) to the red formazan derivative by mitochondrial succinate dehydrogenase in viable cells. 2×104 cells/100 μLin each well of 96 well plates were seeded. Twenty-four hours after cell seeding, cells were incubated with varying concentrations of substance×for 24 hours at 37° C. Following the removal of substance from each well, cells were washed in phosphate buffered saline. The cells were then incubated in serum free DMEM to which MTT (0.5 mg/mL) was added to each well (100 μL), and incubated for a further four hours. Then the medium was removed and the cells are incubated for 15 minutes with 100 μL of acidic isopropanol (0.08 N HCl) to dissolve the formazan crystals. The absorbance of the MTT formazan is determined at 570 nm in an Elisa reader. Viability was defined as the ratio (expressed as a percentage) of absorbance of treated cells to untreated cells.

To evaluate cytotoxic effects of the RNX003 compound, cells were incubated with varying concentrations of the RNX003 compound for 24 h. Following the removal of the RNX003 from each well, cells were washed in phosphate buffered saline, and the MTT assay was carried out as described.

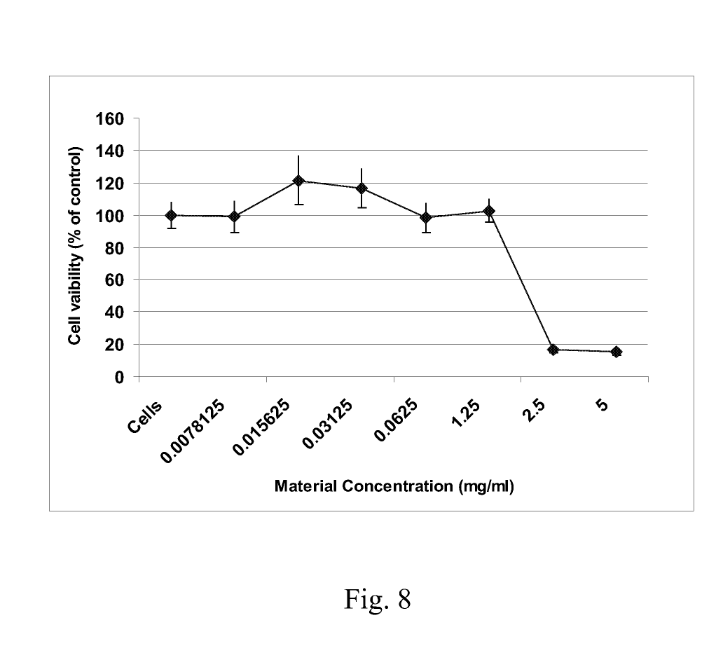

Reference is now made to FIG. 8 showing a chart wherein cell viability is plotted versus the increasing concentrations of the RNX003 compound. No considerable toxic effect was seen after treatment with concentrations up to up to 1.25 mg/ml. Cytotoxic effects (reduction of the number of viable cells) were observed at concentrations higher than 1.25 mg/ml. LD50, is a cytotoxic concentration leading to reduction of 50% of the cell number compared to untreated cells, was found at concentration of the RNX003 compound of about 2 mg/mL.

As envisaged from the empirical data of in-vitro and in-vivo studies elaborated supra, exemplary conjugates, in accordance with the present invention, are characterized by the ability to selectively inhibit the activity of COX-2 and consequently to reduce the effect of a carrageenin-induced edema in the hind paw of rats and decrease PGE2 production and TNFα levels in collagen induced arthritis (CIA) in mice. Furthermore, selected compound is found not to be cytotoxic at the concentrations pertinent to induce a desired therapeutic effect and hence suitable for pharmaceutical applications.

It will be appreciated that the present invention is not limited by what has been particularly described and shown hereinabove and that numerous modifications, all of which fall within the scope of the present invention, exist. Rather the scope of the invention is defined by the claims which follow:

1. A method for treating pain and/or an inflammatory disease or disorder comprising the step of administering a conjugate comprising hydroxyproline covalently linked to a hydrophobic moiety selected from linolenic acid, a-lipoic acid, oleic acid, linoleic acid, arachidonic acid, eicosapentaenoic acid (EPA) and docosahexaenoic acid (DHA), through an amidic bond therebetween.

2. The method of claim 1, wherein the conjugate has the structure according to formula set forth in formula (I):

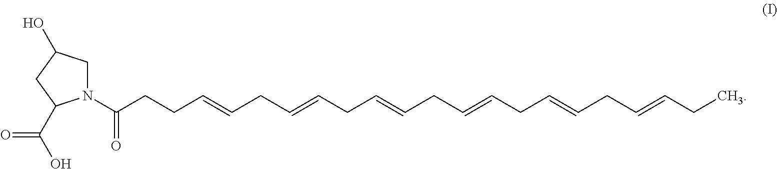 [SMILES: CC/C=C/C/C=C/C/C=C/C/C=C/C/C=C/C/C=C/CCC(=O)N1CC(O)CC1C(=O)O]

3. The method of claim 1, wherein the conjugate is a selective inhibitor of COX-2 relative to COX-1.

4. The method of claim 1, wherein said inflammatory disease or disorder is selected from rheumatoid arthritis, inflammatory bowel disease such, colitis, Crohn's disease, asthma, an autoimmune disease, chronic inflammation, chronic prostatitis, glomerulonephritis, hypersensitivities, pelvic inflammatory disease, reperfusion injury, transplant rejection, vasculitis, diabetes, cardiovascular disorder, and pathogenically induced inflammations.

6. A method of treating cancer comprising the step of administering a conjugate comprising hydroxyproline covalently linked to a hydrophobic moiety selected from linolenic acid, a-lipoic acid, oleic acid, linoleic acid, arachidonic acid, eicosapentaenoic acid (EPA) and docosahexaenoic acid (DHA), through an amidic bond therebetween,

7. The method of claim 6, wherein said cancer is selected from colon cancer, esophagus cancer, lung cancer, bladder cancer, breast cancer and prostate cancer.

8. A conjugate comprising hydroxyproline covalently linked to a hydrophobic moiety selected from an arachidonic acid or eicosapentaenoic acid (EPA).
